(19) United States
(12) Patent Application Publication
(10) Pub. No.: US 2004/0029244 A1
(43) Pub. Date: Feb. 12, 2004

(54) Phospholipase d effectors for therapy and screening

(76) Inventor: Ben-Tsion Williger, Kiryat Ono (IL)

Correspondence Address: Eitan Pearl Latzer & Cohen Zedek, Suite 1001, 10 Rockefeller Plaza, New York, NY, 10020, US

(21) Appl. No.: 10/296,503

(22) PCT Filed: May 24, 2001
(86) PCT No.: PCT/IL01/00481

(51) Int. Cl. C12N 9/99

(52) U.S. Cl. 435/184

(57) ABSTRACT

The pathophysiology of cancer includes the action of matrix metalloproteinases (MMP) enzymes which enable the growth and spread of tumors both locally and as metastatic disease. In the present invention, these proteins are the target for inhibition employing in therapeutic administration phospholipase D inhibitor compounds, some potent compounds being short chain primary alcohols, in particular 1-butanol and 1-propanol. The anti-cancer action of these compounds derives from several pharmacological properties, including the ability to suppress phospholipase D, and both the intracellular and extracellular elaboration of MMP's. Thus disclosed herein are methods for treating a subject afflicted with a cell proliferative disorder, such as colorectal adenocarcinoma, malignant glioma, neuroblastoma, non-small cell lung carcinoma, and breast cancer, comprising the steps of administering an effective amount of 3 phospholipase D inhibitor to a subject in need of such treatment. In addition, provided herein are diagnostic methods for assessing disease activity and tissue sensitivity to treatment.

### FIELD OF THE INVENTION

The present inv ntion provides methods for the identification and treatment of disease states in which action of the enzyme phospholipase D (PLD) plays a role in the pathophysiology. Phospholipase D, as reported here, is a component in the pathway of activation of matrix metalloproteinases, the level of expression of which is linked to a wide variety of diseases, including cell proliferative disorders, invasive cancer and demyelinative disorders. Phospholipase D inhibitors, in particular aliphatic primary alcohols, suppress the level of MMP expression, which thus affords their use as drugs in the treatment of disease, in particular cell proliferative disorders in which MMP levels, in particular that of MMP-9, are pathological. The biological association between PLD and MMP also serves in the design of detection of screening methods for the presence of disease and for the identification of anti-disease compounds.

### BACKGROUND OF THE INVENTION

Phospholipase D is an enzyme found in virtually every type of cell in mammals, known to occur in at least two major isozyme forms, PLD1 and PLD2. The enzyme occurs in both intracellular and membrane-associated forms and in addition to PLD1 and PLD2, at least one additional isozyme form of the enzyme is presumed to exist. PLD1a is expressed mainly in the kidney, small intestine, colon, and liver. PLD1b is mainly found in the lung, heart, and spleen. PLD2 is expressed in almost every tissue and cell types studied. While many of the physiological roles of PLD remain to be discovered, it is now established that PLD1 participates in the vesicular movements associated with intracellular and extracellular organization whereas PLD2 is involved in the Raf-1 pathway. The PLD1 genebank accession numbers are AAB49031 and U69550. The accession number for PLD2 is BAA19882 and U87557.

Matrix metalloproteinases (MMP's) are a family of metal-dependent enzymes involved in the organization of the macromolecular components of the extracellular space and basement membrane. Elaboration of some extracellularly secreted proteins and enzymes has been demonstrated to be dependent upon the activity of phospholipases, such as PLD, and this has also been implicated to be the case for at least two MMP's, namely MMP-2 and MMP-9.

Phospholipase D is an enzyme which catalyzes the hydrolysis of phospholipids, releasing the polar head group and producing phosphatiditic acid (PA). For example, PLD cleavage of phosphatidylcholine (PC) or phosphatidylethanolamine (PE) produces phosphatidic acid and choline or ethanolamine, respectively. The phosphatidic acid (PA) product of the reaction appears to serve in a second messenger capacity, activating additional processes and enzymes within the cell. The enzyme may also produce the active PA product when acting upon other components of the cell as substrates. While many aspects of the intracellular role of PLD and its PA product are yet to be elucidated, one of the isozymes, namely PLD1, is implicated in the process of intracellular trafficking and extracellular secretion (2-5). The other isoforms of PLD are likely to be characterized as having similar properties and physiological effects within the cell, as may be correlated by further study of the process of PLD activation.

Significant in the study of PLD activity of the cell is that in the presence of a primary alcohol, such as 1-butanol or 1-propanol, the lipid. moiety of the substrate is transferred to the exogenously provided alcohol to form a phosphatidylalcohol product (1). Thus PLD activity, and hence the effects exerted by its active products, are inhibited in the presence of such primary alcohols since the shift from PA to phosphatidylalcohol formation results in an reduction in the amount of PA ultimately available to the cell and its active components which are sensitive to the presence of PA.

Matrix metalloproteinases (MMP's) are a family of enzymes, including, but not limited to, collagenases, gelatinases, lamininases, matrilysin, and stromelysins, which are involved in the degradation and remodelling of connective tissue. These enzymes are found in a number of cell types that are found in or associated with connective tissue, such as fibroblasts, monocytes, macrophages, endothelial cells and metastatic tumor cells. They also share a number of properties, including zinc and calcium dependence, biosynthesis as zymogens, extracellular secretion, and about 40-50% amino acid sequence homology.

MMP's degrade the protein components of the extracellular matrix, i.e., the protein components found in the linings of joints, interstitial connective tissue, basement membranes, cartilage, and the like. These matrix tissue proteins include collagen, proteoglycan, fibronectin and laminin.

Under normal physiological conditions, the expression of the constitutive MMPs is low, and regulated by naturally occurring inhibitors. However, under pathological conditions, such as rheumatoid and osteoarthritis, MMP expression in cartilage is disregulated and results in an over-expression of MMPs which are not controlled by constitutive inhibitors. This condition leads to the loss of matrix tissue proteins.

Cancer is considered to be a disorder in which the physiological regulation of cell differentiation, growth, and spread is disturbed. Of major implication in the promotion of cancer cell growth and spread is the perturbed regulation of matrix metalloproteinase (MMP) enzymes, which are known to enable degradative alterations of the extracellular matrix, thus clearing the path and enabling the growth of abnormally proliferating cells, such as cancer cells, to form tumors, invasive tumors, and invasive metastatic lesions. The lesions of cell proliferative disorders are dependent as well, upon local angiogenesis, a process in which MMP action is also involved.

Thus, when in a state of dysregulation or over-expression, MMP's are capable of rearranging extracellular matrix, an event which is associated with the formation of space-occupying lesions, such as tumors, and enhanced angiogenesis in the affected region, two features critical to the promotion of cancer growth. Inhibition of MMP activity, including suppression of both the intracellular expression, i.e. the biosynthesis of MMP proteins, and the extracellular expression, i.e. the secretion of these degradative enzymes, may therefore be considered sensitive targets for limiting the activities of cancer cells which enable local and metastatic lesion formation.

Secretion of matrix metalloproteinases (MMPs) from cancer cells often occurs at an early stage in the disease process, in particular in carcinogenesis, and MMP expression is an important stage in the mechanism of metastatic spread. MMPs hydrolyze collagen, a major component of the extracellular matrix, and thus enable the invasion of cancer cells from their primary site to the circulation and secondary sites (6,7,8).

Several factors are known to affect the expression of MMP's; among these are the effects of phorbol esters. Various stimuli, including phorbol 12-myristate 13-acetate (PMA), a most potent activator of protein kinase C (PKC), have been shown to induce the secretion of MMP-9 from cancer cells (9) including the human fibrosarcoma HT 1080 cells (10,11). Phorbol esters include a number of related derivative compounds that are known to activate PKC. These compounds are exemplified by phorbol 12, 13 dibutyrate, 12-myristate-13-acetate (PMA). Other PKC activating compounds are diacylglycerols, which are considered to be natural activators, and certain intracellular calcium releasing agents, such as thapsigarin. Diacylglycerols are exemplified by 2-acetyl-1-oleoglycerol (OAG). Structural constraints for diacylglycerol activity in activation of PKC is known in the art and described, in a reference by Ganong, et al., incorporated herein by reference.

In addition to cancer, MMP dysregulation is associated with the pathogenesis of myriad diseases, including rheumatoid arthritis, osteoarthritis, periodontal disease, aberrant angiogenesis, multiple sclerosis, Guillain-Barre syndrome, corneal ulceration, and in complications of diabetes. These disorders share in common a dysfunction or structural defects of the extracellular matrix. Inhibition of MMP expression, especially when employing agents acting at an early or preliminary step in the pathway of MMP expression, is therefore recognized as a good target for therapeutic intervention. MMP-9 has been specifically implicated in the development of colon and breast cancer.

Regulation of gene expression and enzyme formation by PLD has been described previously. Among these enzymes are c-fos and c-myc that control gene expression and cell growth (13,14). Therefore, a role for PLD in both the regulation of normal cell growth and in abnormal, cancer-type cell growth has been suggested before.

Although MMP's are known to be implicated in the mechanism of disease, especially metastatic cell-proliferative disorders, methods based upon a therapy wherein the suppression of these proteins, and thus of disease, is accomplished by the administration of phospholipase D inhibitors, has not herethereto been considered or demonstrated in the prior art.

### SUMMARY OF THE INVENTION

The present invention provides methods for the diagnosis and therapy of disease and in screening methods for the screening and identification of anti-disease agents using PLD inhibitors. Specifically, based upon the association between PLD and the intracellular and extracellular expression of MMP disclosed herein, the invention provides methods for the modification or suppression of MMP expression, methods for the treatment of MMP-associated disorders, methods for the diagnosis of MMP-associated disorders, in particular cell proliferative disorders, and methods for the identification of compounds for use in the modification or treatment of MMP or PLD associated disorders.

Inhibitors of phospholipase D are compounds which inhibit one or more of the enzymatic reactions carried out by phospholipase D, an enzyme which preferably hydrolyzes phospholipids, such as phosphatidylcholine to produce choline and phosphatidic acid (PA) or phosphatidylethanolamine to ethanolamine and PA, depending upon the particular isozyme form of PLD. Phosphatidic acid itself may be demonstrated to promote the expression of MMP within the cell or to promote the secretion of MMP into the extracellular space. Thus, reducing the level of phosphatidic acid via inhibition of phosphatidic acid-producing sources, such as phospholipase D, serves to attenuate the expressed level of MMP proteins or zymogens as gene transcripts, secreted proteins, or MMP enzymatic activity of the cell thereof, particularly tissues comprising cancer cells, wherein the expression of MMP levels are abnormally high.

One class of MMP inhibitors, as disclosed herein are inhibitors of phospholipase activity, in particular inhibitors of phospholipase D. Disclosed herein is the ability of primary alcohols, in particular 1-propranol and 1-butanol, to inhibit both the level of MMP expression and the activity of phospholipase D.

While the invention demonstrates the use of phospholipid D inhibitors, the principle upon which the invention is based, namely the association between PLD activity and the level of MMP protein synthesis products, or the association between PLD activity and the level of extracellular elaboration of MMP proteins, does not exclude the use of other effectors of PLD activity to modify MMP expression, as for example, compounds which activate PLD, to achieve similar results.

Thus, in one embodiment of the invention there are provided methods of modifying or suppressing the level of MMP expression of a cell comprising the steps of contacting a cell with an effective amount of a phospholipase D inhibitor, thereby reducing the level of MMP expression of said cell. The cells for which the invention is particularly applicable is a cell characterized by a cell tissue type selected from the group consisting of epithelium, colon epithelium, neuroepithelium, glial cells, astrocytes, endotracheal epithelium, and breast epithelium. When diseased tissue is the object of the invention, the cell is characterized by a cancer cell tissue type selected from the group consisting of colorectal adenocarcinoma, malignant gliomas, neuroblastoma, non-small cell lung cancer, and breast cancer.

In one embodiment of the invention, the phospholipase D inhibitor is a compound comprising at least one primary hydroxyl group or at least one primary sulfhydryl group conjugated to a physiologically acceptable moiety through a linear spacer group n carbon or n heteroatoms atoms in length wherein n is an integer from 3 to 20.

Thus provided by the invention is a method for identifying effector compounds of PLD activity, comprising the steps of: selecting cells from a cell type which expresses MMP or which may be induced to express MMP; contacting said cells with an inducing agent of MMP expression; determining the level of MMP expression of said cells; contacting said cells with a compound initially unknown to affect PLD activity; determining the level of MMP expression of said cells; comparing the determination made in antecedent steps with the determination in subsequent steps; identifying a compound as an effector compound of PLD activity based on at least a 1% difference as determined among the steps.

In another embodiment of the invention, the PLD inhibitor is a serine protease inhibitor.

In another embodiment of the invention, the PLD inhibitor is 4-(2-aminoethyl)-benzenesulfonyl fluoride.

The invention provides further embodiments for a method for identifying effector compounds of MMP expression of a cell, comprising the steps of selecting cells from a cell type which expresses MMP or which may be induced to express MMP; contacting said cells with an inducing agent of MMP expression; determining the level of MMP expression of said cells; contacting said cells with a phospholipase D inhibitor or activator compound; contacting said cells with a compound unknown to affect MMP expression; determining the level of MMP expression of said cells; comparing the determination made in antecedent steps with the determination in subsequent steps; identifying a compound as an effector compound of MMP expression based on at least a 1% difference as determined among the steps.

The above screening or identification methods of the invention may be applied by incorporation of any cell type within the method, including animal, plant, or microbial cells, whether arising from intact tissues, diseased organisms or tissues, or cells modified for use ex vivo including cells in suspension, cultured cells, immortalized cells, cells known to carry or express modified genes or proteins. Particularly preferred for use in the invention are cells wherein the level of MMP expression is constitutive or modified by exposure to reaction conditions or agents which are modifiers of MMP expression. Particular preferred inducing agents of MMP expression are procancerous agents such as phorbol ester compounds, natural effectors such as diacylglycerols, or modifiers of intracellular calcium such as thiagrabsin.

An additional object of the invention, based upon the above methods of identification is to provide compounds or compositions which are useful for the modification or suppression of PLD activity or the level of MMP expression of a cell. Such compounds or compositions identified by the methods of the invention are expected to have benefit in the treatment or diagnosis of disease wherein the pathophysiology comprises the association between a phospholipase and a extracellular matrix altering component, such as the association between PLD activity and MPP expression.

The invention further provides a method for the diagnosis of disease or the propensity to develop a disease, based upon the disclosed association between phospholipase D and MMP expression. Thus provided is a method of identifying a cell or biological tissue exhibiting aberrant levels of MMP expression comprising the steps of: selecting cells from a cell or tissue type which expresses MMP or which may be induced to express MMP; contacting said cells with an inducing agent of MMP expression; determining the level of MMP expression of said cells; contacting said cells with a phospholipase D inhibitor or activator compound; determining the level of MMP expression of said cells; determining the level of MMP expression of said cells or said biological tissue preparation based on comparing the determinations performed in the several steps; identifying a cell or tissue as exhibiting aberrant levels of MMP expression based on at least a 1% difference as determined among the steps.

The present invention further provides a method for treating a subject afflicted with a disorder associated with aberrant levels of PLD activity or of MMP expression, comprising the steps of administering to a subject an effective amount of a phospholipase D inhibitor, thereby treating the subject afflicted with a disorder of PLD activity or MMP expression.

Thus the invention provides a method for treating a subject afflicted with a cell proliferative disorder, comprising the steps of administering to a subject an effective amount of a phospholipase D inhibitor, thereby treating the subject afflicted with a cell proliferative disorder. Thus, in one embodiment of the invention, a phospholipase inhibitor is administered to a subject diagnosed with a cell proliferative disorder as a therapy for attenuating cancer cell growth and spread, thereby treating the subject with a cell proliferative disorder, solid tumor, or metastatic cancer.

Further provided by the invention is a method for treating a subject afflicted with a demyelinative disorder, comprising the steps of administering to a subject an effective amount of a phospholipase D inhibitor, thereby treating the subject afflicted with a demyelinative disorder.

A particularly preferred implementation of the methods of treating a subject afflicted with disease are one in which the phospholipase D inhibitor is a compound which suppresses the level of MMP expression of a cell.

For the purpose of implementing the methods of the invention, the PLD inhibitor or effector compound may be a compound of any chemical structure. Preferred embodiments of the method comprise the use of PLD effectors comprising at least one primary hydroxyl or at least one primary sulfhydryl group conjugated to a physiologically acceptable chemical moiety through a linear spacer group n carbon atoms or n heteroatoms atoms in length wherein n is an integer from 3 to 20. Particularly preferred embodiments comprise the use of compounds selected from the group consisting of 1-propanol, 1-butanol, 1-propanthiol, 1-butanthiol, or mixtures thereof. Wherein the PLD effector employed is conjugated to a physiologically acceptable chemical moiety, said moiety may be any atom or chemical group. In these specific embodiments, the chemical moiety may be any atom or chemical group which serves to enhance the efficacy of the so-conjugated PLD inhibitor, whether through enhancing stability, permeability, solubility, or biological efficacy of the PLD inhibitor or effector. In further preferred embodiments, the conjugated chemical moiety is in itself an effector of phospholipase D activity or of MMP expression.

The methods of the present invention are directed in particular to the use of method of cells selected from the group consisting of epithelium, colon epithelium, glial cells, astrocytes, endotracheal epithelium, breast epithelium, or to the use of diseased cells selected from the group consisting of colorectal adenocarcinoma, malignant gliomas, neuroblastoma, non-small cell lung cancer, and breast cancer.

In any particular embodiment of the invention, the said desired suppression of MMP levels may be achieved through attenuation, in particular, of MMP-9 levels.

### BRIEF DESCRIPTION OF THE FIGURES

FIG. 1. PMA induces MMP-9 secretion in HT 1080 cells. A, overnight serum-deprived cells were incubated in freshly added DMEM, 0.1% BSA. Cells were exposed to the phorbol ester PMA (100 nM) for the times indicated. Dimethyl sulfoxide (DMSO) solvent was used as the control for PMA. After 7.5 h, medium samples (5 [symbol] l) were separated on 10% zymogram gels, and MMP-9 activity was assayed as described under “Methods and Materials”. B, cels were incubated with the indicated concentrations of PMA. After 7.5 h, medium samples were assayed as above for MMP-9 activity. C, cells were preincubated with or without 5 [symbol] M PKC inhibitor Ro 31-8220 for 1 h and then either PMA (100 nM) or DMSO control were added. Following 7.5 h, medium samples were assayed as above for MMP-9 activity. The data shown for all panels are representative of three independent experiments.

FIG. 2. Activation of PLD in PMA-treated HT 1080 cells. The cells, at their log phase of growth, were serum deprived and labeled with 1 z,900 Ci/ml [³H]myristic acid. Following washing and preincubation with DMEM, 0.1% BSA, 0.3% 1-butanol for 20 min, and 100 nM PMA were added, and cells were incubated for the indicated times at 37° C. Lipids were extracted and analyzed, and the production of labelled phosphatidylbutanol ([³H]PtdBut) was measured as described under “Materials and Methods”. The values obtained for [³H]PtdBut were normalized by dividing the measured counts/min by counts/min in the total lipid fraction. Data are expressed as the mean±S.E. of the fold activation in three independent experiments performed in triplicates.

FIG. 3. MMP-9 secretion in response to DDPA treatment. Overnight serum-deprived cells were incubated in freshly added DMEM, 0.1% BSA with the indicated concentrations of diodecanoylphosphatidic acid (DDPA, or “DOPA”). After 7.5 h, media samples (5 [symbol] l) were assayed for MMP-9 activity as described under “Materials and Methods”. The data represent three independent experiments.

FIG. 4. MMP-9 secretion is inhibited by 1-propanol. Overnight serum-deprived cells were incubated in freshly added DMEM, 0.1% BSA. A, the indicated concentrations of either 2-propanol or 1-propanol were then added. After 45 min, 100 nM PMA-was added and cells were incubated for 7.5 h after which media samples (5 [symbol] l) were analyzed for MMP-9 secretion. B, the cells were incubated with 133 mM 2-propanol or 1-propanol for 45 min. PMAa was then introduced to give the indicated concentrations. The cells were incubated for 7.5 h after which 5 [symbol] l media samples were analyzed for MMP-9 secretion. The data shown are representative of three independent experiments.

FIG. 5. Actinomycin D inhibits MMP-9 secretion in HT 1080 cells induced with Dhosphatidic acid. Overnight serum-deprived cells were incubated in freshly added DMEM, 0.1% BSA, 10 [symbol] g/ml ActD for 45 min and then induced with 80 [symbol] g/ml DDPA Following 7.5 h incubation 5 [symbol] l samples were assayed for MMP-9 activity as described under “Experimental Procedures”. The data shown are representative of three independent experiments.

FIG. 6. Phophatidic acid mediates protein kinase C induction of MMP-9 expression. A, Overnight serum-deprived cells were incubated in freshly added DMEM, 0.1% BSA containing either 100 nM PMA or 80 [symbol] g/ml DDPA. B. Overnight serum-deprived cells were incubated in freshly added DMEM, 0.1% BSA containing 0.5% of either 1-propanol or 2-propanol for control. Following 7.5 h incubation cells were lysed with RIPA buffer and 15 [symbol] g whole cell lysate were subjected to SDS-PAGE and immunoblotting with rabbit anti MMP-9. Immunoreactive bands were visualized using the ECL reaction. The data shown are representative of three independent experiments.

### DETAILED DESCRIPTION OF THE INVENTION

A role for PLD in both the regulation of normal cell growth and in abnormal, cancer-type cell growth has been suggested before (13, 14) although the use of PLD inhibitors in screening methods for drug discovery and as specific therapeutic agents has not heretofore been disclosed. The pr sent invention discloses that PLD activation is a critical step in the expression of MMP's, a family of proteins involved in a wide variety of pathological disease states. Thus the present invention is based upon the ability of inhibitors of PLD to modify or suppress the level of MMP expression.

Definitions: The term phospholipase D (PLD) refers herein to all isozyme forms of the protein, including PLD1 and PLD2, which display the enzymatic or biological activity of phospholipase D, and which are intracellular or localized to cell membranes. The enzymatic activity of PLD is defined by the hydrolysis of polar phospholipids to produce phosphatidic acid (PA) and a free polar head group. Different isozymic forms of PLD hydrolyze preferably phosphatidylcholine to produce PA and choline or phosphatidylethanolamine to produce PA and ethanolamine. The biological activity of PLD is defined herein both by the enzymatic action on cell substrates and to the physiological changes affected by the products of the enzyme. Thus, given the ability of PLD to produce PA, and to affect MMP expression, the term PLD refers to these biological activities as well.

The terms “MMP expression” or “level of expression” refer herein to any one of the several biological expressions of this class of proteins known to occur within and around cells, including the rate of production or secretion by a cell of MMP gene transcription protein products, MMP zymogens or pro-proteins, MMP proteins, MMP protein fragments, MMP secreted proteins, MMP enzymatic activity, antigenic determinants of MMP proteins or fragments thereof. For purposes of the invention, MMP expression refers to both intracellular and extracellular forms of MMP proteins, independent to, but including, enzymatically active forms.

An “inducing agent” is any compound of composition which modifies the rate of MMP or PLD expression of a cell, acting at either the stage of the expression of proteins or fragments thereof, including protein synthesis, protein folding, post-translational modification of protein, protein sequestration or protein secretion, such that said expression is enhanced. An inducing agent of MMP is any substance or compound which enhances the rate of MMP proteins, enzymes, isozymes, zymogens, or fragments thereof produced by a cell and amenable to detection by immunological or enzymatic means whether within whole cells, disrupted cells, or within the extracellular milieu. An inducing agent of PLD is any substance or compound which enhances the rate of PLD proteins, enzymes, isozymes, zymogens, or fragments thereof produced by a cell and amenable to detection by immunological or enzymatic means whether within whole cells, disrupted cells, or within the extracellular milieu. Specific examples of MMP inducing agents for use in the methods of the invention are activators of protein kinase C activity, such as phorbol esters, diacylglycerols, thapsigarin, or PA. MMP inducing agents may or may not act as inducing agents of PLD. Said inducing agents may be applied as agents administered in situ, in vitro, or in vivo, depending on the cell type or location chosen for implementation of the invention and its methods.

Suppressing the level of MMP expression refers to reduction in the level of MMP enzymes, proteins, pro-proteins, zymogens, or fragments thereof, wherein the level or rate of synthesis, transcription, post-transcription processing or secretion by a cell is altered upon exposure to an agent or compound in comparison said level or rate in the absence of said agent or compound.

“A physiologically acceptable conjugated moiety” is any atom or chemical group which is capable through covalent attachment to modify a PLD effector compound, whether an inhibitor or an activator of the enzyme, to enhance the chemical stability, physiological stability, permeability, affinity, or solubility of said effector. For purposes of the invention, a physiologically acceptable conjugated moiety may serve as a reporter group, whether incorporating a radioactive or other form of group amenable to conventional detection methods. Examples of physiologically acceptable conjugated moieties are atoms or chemical groups selected from the list comprising hydrogen, halogens, hydroxyl, sulfhydryl, amino, cyano, nitro, phosphate, thiophosphate, mercapto, lower alkyl, lower alkenyl, aromatic rings, heterocyclic rings, heterocyclic aromatic rings, carboxyl, cycloalkyl, cycloalkylalkyl, alkyloxycarbonylalkanoyl, alkyloxycarbonyl, alkanoyl, cycloalkylcarbonyl, heterocycloalkylcarbonyl arylalkyloxycarbonyl, carbamoyl, alkylcarbamoyl, dialkylcarbamoyl, arylcarbamoyl, arylalkylcarbamoyl, arylalkanoyl, aroyl, alkylsulfonyl, dialkylaminosulfonyl, arylsulfonyl, heteroarylsulfonyl, alkyl cycloalkyl, cycloalkylalkyl, heteroaryl, heteroarylalkyl, aryl, arylalkyl, alkoxy, alkylsulfonyl, an arylsulfonyl, saccharides, polysaccharides, glycosaminoglycans, salicylates, steroids, hydroxysteroids, purines, pyrimidines, nucleosides, amino acids, peptides, glycerides, poly-glycerides, glycols, polyglycols, lipids, individual isomers and combinations thereof.

“A linear spacer group n carbon atoms in length” is a linear alkyl or alkenyl chain n carbon atoms in length comprising up to three carbon-carbon double bonds, and up to 15 hydrogen atoms, wherein n is an integer ranging from 3 to 10.

“A linear spacer group n heteroatoms in length” is a linear chain of n atoms in length comprising up to six double bonds, wherein said atoms are selected from the group consisting of carbon, oxygen, nitrogen, or sulfur, and said atoms of said chain are bound in addition to at least one hydrogen atom, and wherein n is an integer ranging from 2 to 20.

Effector compounds are either inhibitors or activators of a biological process of a cell whereupon contact of the cell with an agent or compound results in an increase or reduction in the rate of said biological process in comparison to the rate in the absence of said effector. A biological process may be the gene transcription or other post-transcriptional stages of protein synthesis, protein or enzyme secretion by the cell, or enzymatic activity of a protein within said cell or within the extracellular medium of said cell.

Levels of MMP expression are aberrant when either the intracellular or extracellular amount of MMP protein material of a cell, are different from the amount of MMP protein material expected to be found for a similar cell from a healthy subject.

MMP protein material of a cell is proteins, pro-proteins, zymogens, or fragments thereof are assayed by immunological or enzymatic methods, which are specific for identifying and quantifying MMP's.

Lev Is of PLD expression are aberrant when either the intracellular or extracellular amount of PLD protein material of a cell, are different from the amount of PLD protein material expected to be found for a similar cell from a healthy subject.

PLD protein material of a cell is proteins, pro-proteins, zymogens, or fragments thereof are assayed by immunological or enzymatic methods, which are specific for identifying and quantifying PLD.

“A compound initially unknown to affect PLD activity” is any compound which is not previously known through direct enzymatic assay of PLD activity wherein a known substrate of PLD is employed to either inhibit or activate the rate of the enzymatic reaction of PLD.

“A compound initially unknown to affect MMP expression” is any compound which is not previously known to affect the level of expression of the MMP content of a cell when tested through direct immunological or enzymatic assay specific for MMP protein material.

MMP suppression is not only an important target in treating diseased tissue but, when measured alone or in combination with relevant inducers or inhibitors, is also an excellent target for identifying new drug modalities, as therapeutic compounds or compositions, and for diagnostic testing of tissue samples. Diagnostic testing based upon the interrelationship between PLD activity and MMP expression is a new modality providing opportunity for both early detection of disease, thus allowing for earlier intervention, and for estimating the potential of primary loci of malignant cells to manifest invasiveness, thus advancing characterization of the disease state and hence serving to facilitate the clinical decision process. Levels of MMP expression are aberrant when either the intracellular or extracellular amount of MMP protein material of a cell, are different from the amount of MMP protein material expected to be found for a similar cell from a healthy subject.

Levels of MMP expression are aberrant when either the intracellular or extracellular amount of MMP protein material of a cell, are different from the amount of MMP protein material expected to be found for a similar cell from a healthy subject.

MMP protein material of a cell is proteins, pro-proteins, zymogens, or fragments thereof are assayed by immunological or enzymatic methods, which are specific for identifying and quantifying MMP's.

Levels of PLD expression are aberrant when either the intracellular or extracellular amount of PLD protein material of a cell, are different from the amount of PLD protein material expected to be found for a similar cell from a healthy subject.

PLD protein material of a cell is proteins, pro-proteins, zymogens, enzymes, isozymes, or fragments thereof are assayed by immunological or enzymatic methods, which are specific for identifying and quantifying PLD.

Thus the invention herein provides for methods wherein the association between PLD and MMP's is subjected to testing in assay systems which are designed to measure these interrelated cellular events in combination as an index of the pathological state of a cell, group of cells, or tissue sample. These methods may be as well readily adapted to the diagnosis of MMP-related disease states of organs, parts of organs, or within a living animal.

Another aspect of the invention is to provide a means for identifying compounds or compositions of therapeutic value based on the ability to affect PLD activity as measured by the resulting effect on the level of MMP expression. Thus, any compound may be tested as a potential agent in disease pathogenesis associated with aberrant MMP expression. Compounds to be tested may be chosen at random or selected for testing in the assay system provided by the invention based upon selection at random or selected based upon structural or pharmacological evidence suggesting an ability to modify PLD activity. The assay methods may be employed for testing individual compounds or readily adapted for the screening of mixtures of compounds, as may occur when testing the products of combinatorial chemistry synthetic methods. Inhibitors of PLD activity are expected to suppress MMP expression and thus manifest anti-disease effects. In contrast, the methods employed may also serve to identify compounds which activate PLD as manifested through enhanced levels of MMP expression. This later aspect of the screening process directs the user of the invention to identify agents which potentially promote disease through MMP-related mechanisms, and thus may serve to characterize the noxious potential of agents known or unknown to produce disease, in particular carcinogenic substances. The pharmacological properties of compounds or -compositions-identified by the assay system described herein may be subject to further stages of characterization, for example by the use of direct in vitro, in situ, or in vivo assays of PLD activity, wherein appropriately rate of change in the level of labeled phospholipid substrates or of the reaction products of PLD activity form the basis of the assay parameters to be measured and assessed through conventional or modified assay techniques for phospholipases.

In addition to the identification of effectors of PLD activity, the invention provides for additional modifications of the assay system wherein the incorporation of additional test compounds provides a means for the identification of additional suppressor or activators of MMP expression. In this aspect of the invention, the concomitant use of PLD inhibitors or activators provides for a means of manipulating the inter-relationship between PLD activity and MMP in a way which provides for controlling for varying levels of PLD activity, thereby allowing for the assessment of the effects of compounds or compositions which effect the level of MMP expression through additional physiological pathways, and thus enabling for identification of anti- or pro-disease agents which act in tandem or independent to the relationship between PLD activity and the level of MMP expression. Thus, for example, a compound may be identified to exert effects on MMP expression under assay conditions chosen to obliterate or substantially modify the level of PLD activity as may obtained through inclusion of known PLD effector compounds in the assay process. This aspect thus affords the user of the invention the opportunity to identify compounds or compositions which affect MMP expression, in whole or in part, through physiological mechanisms independent to or in conjunction with the level of PLD activity of the cell. Agents identified by this method may then be applied to the modification of MMP expression alone or as adjunctive for therapy or screening for MMP-related disease.

Specifically, it is disclosed herein that PLD activity mediates the cellular effect of MMP-inducing agents. More specifically, it is disclosed that inhibitors of PLD suppress the effect of upstream regulators, such as PKC, on th level of MMP-9 expression in HT 1080 cells. Inhibition by PLD inhibitors, such as primary alcohols, of the PA-producing enzyme PLD has a multiple of pharmacological effects including depriving the cell of PA, suppressing the intracellular expression of MMP-9 as a product of the process of gene transcription, and suppressing the extracellular level of MMP-9. Since MMP-9 is a major enzyme associated with the promotion of angiogenesis and the invasiveness of cancer cells at both primary and secondary sites, this finding demonstrates that inhibition of PLD can block tumor growth and metastases formation. Thus the data presented herein establish that the use of PLD inhibitors, such as primary alcohols, are of substantial benefit in the treatment of a subject afflicted with a cell proliferative disorder characterized by uncontrolled or invasive cell growth associated with MMP dysregulation, such as is known to occur in solid tumors of malignant potential and in metastatic cancer.

In mammal tissues, the activity of matrix metalloproteinases is highly regulated. As a result, the breakdown of connective tissue mediated by these enzymes is generally in a dynamic equilibrium with the synthesis of new matrix material. In a number of pathological conditions, however, dysregulation of MMP activity leads to the uncontrolled breakdown of extracellular material. Inhibitors of MMP's thus are expected to provide useful treatments for diseases associated with the excessive degradation of extracellular matrix as described by examples below.

However, given the wide variety of MMP-related diseases, the utility of PLD inhibiting drugs is not necessarily limited to the context of anti-cancer therapies but rather is anticipated to alleviate the signs or symptoms of the group of diseases, known in general as collagen or inflammatory disease, wherein disruption of the extracellular matrix is a prominent feature, including rheumatoid arthritis, osteoarthritis, periodontal disease, gastric ulceration, corneal ulceration, psoriasis, multiple sclerosis and Guillian Barre Syndrome. In addition, other MMP-related diseases likely to respond to a PLD inhibitor mode of therapy are aberrant angiogenesis syndromes, complications of diabetes, HIV infection, and bone dis ase, among others. Some MMP's directly implicated in the pathogenesis of disease are MMP-9 and MMP-2.

The present invention provides for the use of PLD inhibitors and their physiological effects, among which is the inhibition of MMP expression. Thus, in contrast to compounds which inhibit MMP activity, PLD inhibitors are particularly useful in that the desired pharmacological action is exerted at early stages in the pathway of MMP extracellular action, namely suppression of the level of MMP expression at both the levels of protein translation and protein secretion. As demonstrated in the Examples below, the inducing agent PMA stimulates both MMP-9 secretion and PLD activity in a time and dose dependent manner. Inhibition of PA production by primary alcohol blocks MMP-9 secretion. Furthermore, addition of diodecanoylphosphatidic acid (DDPA; short chain analogue of PA) induced high secretion of MMP-9.

Further study of the effect of PA reveals that PA induces de novo MMP-9 expression in HT 1080 cells and inhibition of its production blocks or suppresses MMP-9 expression. Also, inhibition of PA accumulation results in suppressed or inhibited MMP-9 expression in PMA-treated cells. Moreover, exposing the cells to actinomycin D (ActD), a specific inhibitor of protein translation, prevents the PA-stimulation of MMP-9 secretion. Taken together, the data presented below in the Examples herein show that PA, a direct product of PLD activity, is a major mediator of MMP-9 expression and secretion upon activation, such as by PKC. In particular, it is shown that inhibition of PA accumulation in cells blocks both the production of MMP-9 and of its secretion. Therefore, administration of PLD inhibitors, such as primary alcohols, which inhibit MMP-9 expression and secretion, comprise an effective method for the suppressing the level of MMP expression and may therefore be considered to be of value in the treatment of MMP-associated diseases. Finally, MMP-9 secretion is a characteristic of many cancer types such as colorectal adenocarcinoma (15), malignant gliomas (16), neuroblastoma (17), non-small cell lung cancer (18) and breast cancer (19). For purposes of further description, the publications cited below are included for reference.

Other PLD inhibitors include some compounds which are also inhibitors of serine proteases. A serine protease is a hydrolytic enzyme which has a serine residue at its active site and cleaves peptides or proteins. In some cases, serine proteases also cleave esters. An example of a serine protease inhibitor which is also an inhibitor of PLD is the compound 4-(2-aminoethyl)-benzenesulfonyl fluoride (20). This compound is a polar compound and of low permeability to biological membranes, such as lipid bilayers, cell membranes, mucosa, gastrointestinal lining, kidney, tubules, or blood-brain barrier. According to the invention, PLD inhibitors are conjugated to a physiologically acceptable moiety to enhance the chemical stability of the inhibitor, physiological stability of the inhibitor, cell membrane permeability of the inhibitor, or a combination thereof. According to the invention, conjugating a serine protease inhibitor which is also a PLD inhibitor to a physiologically acceptable moiety has the advantage of achieving a greater inhibition of intracellular PLD activity, wherein the conjugated moiety is a lipophilic or essentially hydrophobic group which enhances the permeability of the inhibitor moiety to a biological membrane, such as a lipid bilayer, a cell membrane, a mucosa layer, the gastrointestinal mucosa, the kidney tubule, the blood-brain barrier, or a combination therof.

Thus, according to the invention, said phospholipase D inhibitor is conjugated to a physiologically acceptable moiety through a linear spacer group n carbon or n heteroatoms atoms in length, wherein n is an integer from 2 to 20, wherein said spacer group is covalently attached to a nitrogen atom of said inhibitor. According to one embodiment of the invention, said phospholipase D inhibitor is conjugated to a physiologically acceptable fatty acid, lipid, phospholipid or thiophospholipid moiety through a linear spacer group n carbon atoms or n heteroatoms atoms in length, wherein n is an integer from 0 to 20, wherein said spacer group is covalently attached to a nitrogen atom of said inhibitor and to an oxygen, hydroxyl, carboxyl, sulpher, thiosulphyl, phosphate or thiophosphate atom or group of said fatty acid, lipid, phospholipid or thiophospholipid moiety. In another embodiment of the invention, said physiologically acceptable conjugated moiety is an atom or chemical group selected from the list consisting of hydrogen, halogens, hydroxyl, sulfhydryl, amino, cyano, nitro, phosphate, thiophosphate, mercapto, lower alkyl, lower alkenyl, aromatic rings, heterocyclic rings, heterocyclic aromatic rings, carboxyl, cycloalkyl, cycloalkylalkyl, alkyloxycarbonylalkanoyl, alkyloxycarbonyl, alkanoyl, cycloalkylcarbonyl, heterocycloalkylcarbonyl arylalkyloxycarbonyl, carbamoyl, alkylcarbamoyl, dialkylcarbamoyl, arylcarbamoyl, arylalkylcarbamoyl, arylalkanoyl, aroyl, alkylsulfonyl, dialkylaminosulfonyl, arylsulfonyl, heteroarylsulfonyl, alkyl cycloalkyl, cycloalkylalkyl, heteroaryl, heteroarylalkyl, aryl, arylalkyl, alkoxy, alkylsulfonyl, an arylsulfonyl, saccharides, polysaccharides, glycosaminoglycans, salicylates, steroids, hydroxysteroids, purines, pyrimidines, nucleosides, amino acids, peptides, glycerides, poly-glycerides, glycols, polyglycols, lipids, phospholipids, thiophospholipids, individual isomers, and combinations thereof.

In another embodiment of the invention, said physiologically acceptable conjugated moiety is also an inhibitor, an activator, or substrate of phospholipase D. According to this embodiment of the invention, the conjugated moiety serves to enhance the inhibitory effect of the PLD inhibitor moiety through achieving a longer or more avid interaction between the inhibitor and the phospholipase.

According to the invention, conjugation of a PLD inhibitor to a physiologically acceptable moiety may be carried out by any acceptable means including, but not only, chemical synthesis, enzymatic synthesis, or a combination thereof. For purposes of implementing the present invention, conjugated PLD inhibitors are preferably purified prior to use in one of the methods of the invention.

According to the invention, examples of conjugated PLD inhibitors are, but not only, the following:

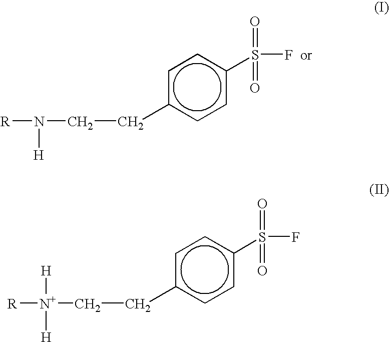

wherein R is a fatty acid, a lipid, a phospholipid, or a thiophospholipid.

According to the invention, examples of conjugated PLD inhibitors are, but not only, the following:

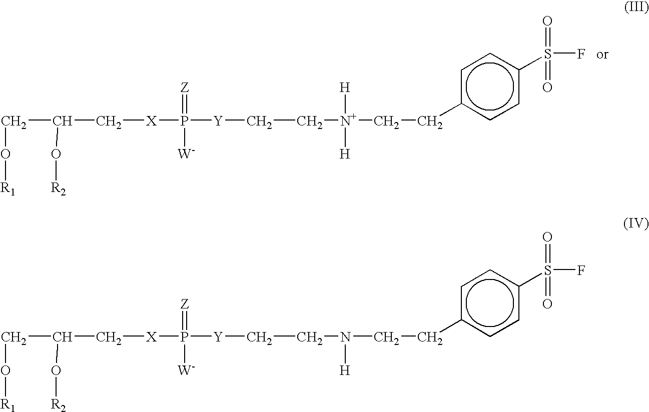

wherein R₁ and R₂ are each independently a hydrogen atom, an acyl group or an alkyl group;

X is oxygen, sulfur, or methylene;

Y is methylene, oxygen, or sulfur;

Z is oxygen or sulfur; and,

W is oxygen or sulfur.

According to the invention, examples of conjugated PLD inhibitors are, but not only, the following:

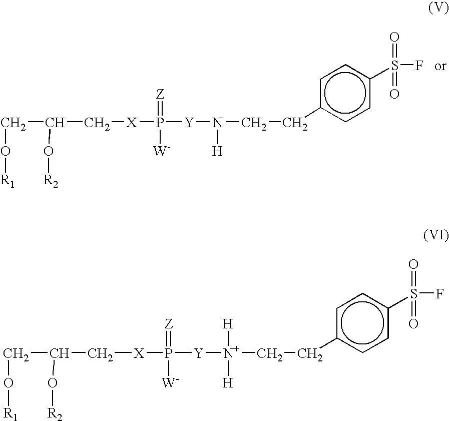

wherein R₁ and R₂ are each independently a hydrogen atom, an acyl group or an alkyl group;

X is oxygen, sulfur, or methylene;

Y is methylene, oxygen, or sulfur;

Z is oxygen or sulfur; and,

W is oxygen or sulfur.

METHODS OF TREATMENT AND ROUTES OF ADMINISTRATION—Primary alcohols, as volatile liquids at room temperature, may be administered in vivo by a variety of alternative routes or combinations thereof. The amount and volume of PLD inhibitor to be administered will mostly depend on clinical considerations, such as the distribution of the disease, the size of disease lesions, the type of tissue or organ affected, the extent of tissue absorption and metabolism of the inhibitor at the site of administration, and the clinical state of the patient.

For systemic administration, compounds such as 1-propanol or 1-butanol may be dissolved in physiologically acceptable solutions, such as water and ethanol, and the resultant pharmaceutical preparation administered intravenously at a rate and in a quantity sufficient to achieve a tissue level effective to suppress the expression or secretion of matrix metalloproteinases, in particular MMP-9. Alternately, the pharmacological efficacy of the remedy may be judged by monitoring the physiological course of the disease itself, such as tumor location, tumor size, tumor growth rate or tumor cell number, extent of tumor vascularity, distribution of metastatic lesions, or other signs of cancer cell activity, such as following the course of a known biochemical marker of the cancer type being treated. An adequate administered dose should provide free plasma levels ranging from 0.002% to 0.5% (w/w). The skilled physician will be able to adjust the dosage with respect to the nature and severity of the condition being treated, the type of tissue involved, and in accordance with the therapeutic index of the drug. For enhanced safety, it is advisable to monitor tissue levels of the administered compounds or their metabolites in the course of treatment. Other PLD inhibitors may be administered in a similar fashion, wherein the dosage and pharmaceutical carrier for intravenous administration would be dictated by the predominating physicochemical and pharmacokinetic properties of the compounds, as one skilled in the art would readily determine.

In addition to systemic intravenous administration, a patient may be treated via routes of local administration, wherein the drug is introduced to the body in a manner which provides for higher concentrations of the drug at the locus of disease. Thus, for treating a patient, a phospholipase D inhibitor may be administered directly via hypodermic injection into a locus of disease, such as a tumor arising from a cell proliferative disorder. Alternatively, the drug may be introduced by catheter delivery into vessels supplying the locus of disease. Such methods may be used in combination with other pharmacological agents, as desired by the physician, including additional anti-disease agents or vaso-occlusive or vasoconstrictive agents which may serve to further contain the local distribution of the drug. Pharmaceutical compositions comprising a PLD inhibitor in combination with physiological acceptable carriers which serve to limit the rate of distribution of the inhibitor, thereby maintaining local concentrations of the drug at higher levels or for more prolonged periods of time would be expected to provide additional advantages as a formula for administration. In particular such carriers are viscous compositions such as physiologically compatible oils or ethylene glycols.

Similarly, the anti-cancer primary alcohol compounds may be administered as adjunctive therapy in combination with other modalities, including chemotherapies and radiation treatment. Dosage adjustments of each modality may be performed in accordance with the experience and skill of the treating physician and the dynamic nature of the condition subjected to treatment.

Enteral administration of PLD-inhibiting drugs, including the primary alcohols, is also a means for delivery of the drug, wherein pharmaceutical preparations comprising one of the active compounds are prepared as capsules, drops, tablets, or suppositories.

Topical administration, in the form of direct application of the active drug or in admixture with a carrier or cream, is a route which may be exploited both for treatment of local or cutaneous lesions, or as a means of achieving effective tissues levels of the active drug through the process of transdermal passage. Topical administration may be preferred by the clinician for the treatment of sites of cell proliferative disorders affecting the skin, such as skin cancer or psoriasis.

Intrathecal administration of PLD inhibitors, when combined with appropriate pharmaceutical carriers and solvents, may be preferred when the site of the lesion is in the central nervous system, such as brain or spinal cord tumors or in demyelinative conditions such as multiple sclerosis.

Similarly, inhalation of the PLD inhibitors, in particular the volatile liquid forms, may be exploited to achieve effective tissue levels, whether directed at lesions within the gastric or respiratory lumens or, in the case of more distal lesions, through the process of transluminal passage to the circulatory system.

Direct administration of the PLD inhibitors, via transdermal needle or intravascular catheter, preferably under the guidance of a suitable imaging technique, is yet another alternative route of administration, wherein the active drug, or pharmaceutical preparations thereof, may be effectively directed toward the site of a tumor or of a vessel supplying a tumor, while providing for control of excessive drug levels which may lead to toxic reactions in non-target tissues.

PROCESS FOR IDENTIFYING NOVEL PLD EFFECTOR COMPOUNDS—The association between PLD activity and MMP expression can be exploited in a screening technique designed to identify novel PLD effector compounds, such as inhibitors and activators. Since PLD activity is a critical component in the pathway of MMP expression, a compound can be tested for an effect on PLD activity through an assay based upon the level of expression of MMP's. Such an assay may be performed in any cell or tissue known to express MMP's constitutively or in a cell or tissue preparation induced to express MMP's through exposure of cells to an agent known to induce MMP expression. Employing an inducing agent of MMP expression allows for the application of a wider range of tissue and cell types to the assay system as well as the use of tissue and cell types which are particularly susceptible to an MMP inducing agent. Achieving higher levels of MMP expression through the use of an inducing agent allows for an assay system of greater sensitivity, both in a quantitative aspect wherein the effect to be measured, i.e. inhibition, may be more easily ascertained, as well as in a qualitative aspect, wherein the assay system may detect both a constitutive and an induced form of MMP expression. Including MMP inducing agents within the assay system allows as well for assessing the effect of a compound on PLD activity wherein the resultant suppression of MMP expression may occur differentially, depending upon whether the level of expression measured is constitutive, induced, or both.

The system for detection of PLD inhibitors provided by the invention comprises several steps, which may be performed in the following order selecting cells from a cell type which expresses MMP or which may be induced to express MMP; contacting said cells with an inducing agent of MMP expression; determining the level of MMP expression of said cells; contacting said cells with a composition unknown to affect PLD activity; contacting said cells with an inducing agent of MMP expression; determining the level of MMP expression of said cells; comparing the determinations made for MMP levels; and identifying compounds as inhibitors of PLD activity based on at least a 1% change in the level of MMP expression. As exemplified below in the Examples, MMP expression may be measured by immunological means, employing antibodies directed against determinants of MMP proteins, zymogens, or fragments thereof, or, alternatively, direct assaying the extracellular medium for MMP enzymatic activity.

Several of the steps may modified as needed to perform controls necessary for identifying inhibitor compounds of PLD. For example, while some of the compounds to be tested, including structural analogues of primary aliphatic alcohols, are likely to exhibit the same or higher potencies of biological activity as PLD inhibitors, it cannot be excluded that some structural analogues will have opposite effects. Thus, in this sense, the screening process provides utility not only for identifying inhibitors of PLD but as well for compounds which may be activators of PLD activity.

The above process for identifying PLD effectors may be applied to any compound or composition which is presumed, based upon its molecular structure, to be a PLD inhibitor or activator. Preferably, as with known PLD inhibitors, i.e. 1-propranol or 1-butanol, the compound to be tested comprises at least one primary hydroxyl group attached to a physiologically acceptable carrier moiety through a linear alkyl chain spacer group n carbon atoms in length wherein n is an integer from 3 to 10. Alternatively, the spacer group may be a linear group n heteroatoms atoms in length wherein n is an integer from 3 to 20. Alternatively, the primary hydroxyl group may be replaced by a chemically analogous group, such as a primary sulfhydryl group.

The carrier moiety of the compound to be tested may be any physiologically acceptable molecular species, including an sulfhydryl, a hydroxyl, an amino, a cyano, a phosphate, a thiophosphate, a mercapto, a lower alkyl, a lower alkenyl, an aromatic ring, a heterocyclic ring, a saccharide, a polysaccharide, a glycosaminoglycan, a salicylate, an amino acid, a peptide, a glyceride, a glycol, a lipid, or combinations thereof.

One skilled in the art and acquainted with the invention and examples disclosed herein, may readily apply the above method for the identification of a compound or composition which is an inhibitor or activator of PLD activity.

PROCESS FOR IDENTIFYING EFFECTORS OF MMP EXPRESSION—The association between PLD activity and MMP expression can also be exploited in a detection method designed to identify novel effector compounds of MMP expression which employing known effectors of PLD activity. Much like the process described above for detecting PLD effectors, this process incorporates the use of cells or tissues which either express constitutive levels of MMP's or which may be induced to express MMP's upon exposure to an appropriate inducing agent. In contrast to the system described above, however, the system described herein employs the use of compounds or compositions which are known to inhibit or activate PLD activity. In addition, further compounds may be introduced into the assay steps to identify compounds which enhance or suppress MMP expression in the presence of PLD effectors. This detection system thus provides for the opportunity to quantify the extent of suppression of MMP expression produced by known inhibitors of PLD as well as to identify compounds which depend upon, or act synergistically to, concomitant PLD inhibition in suppressing MMP expression. Additionally, the system allows for the detection of activators of the level of MMP expression, thus serving as a system to determine the pathological potential of a test substance.

The system for detection of MMP effectors provided by the invention comprises several steps, which may be performed in the following order selecting cells from a cell type which expresses MMP or which may be induced to express MMP; contacting said cells with an inducing agent of MMP expression; contacting said cells with a compound unknown to suppress MMP expression; determining the level of MMP expression of said cells; contacting said cells with a PLD inhibitor; contacting said cells with an inducing agent of MMP expression; contacting said cells with a compound unknown to suppress MMP expression; determining the level of MMP expression of said cells; comparing the determination made of MMP expression in the final step with that of preceding steps to quantify the suppressing or activating a compound previously unknown to affect MMP expression.

Several of the steps may modified as needed to perform controls necessary for identifying inhibitor compounds of MMP expression. For example, while some of the compounds to be tested, including structural analogues of primary aliphatic alcohols, are likely to exhibit the same or higher potencies of biological activity as PLD inhibitors, it cannot be excluded that some structural analogues will have opposite effects. Thus, in this sense, the screening process provides utility not only for identifying inhibitors of MMP expression PLD but as well for compounds which may b activators of MMP expression. Thus, in this sense, the screening process provides utility not only for identifying suppressors of MMP expression but for the possibility of characterizing compounds which may have pathological effects in promoting disease through enhanced MMP expression.

The above process for identifying MMP suppressors and for quantifying PLD inhibitors may be applied to any compound or composition which is presumed, based upon its molecular structure, to be a PLD inhibitor. Preferably, as with known PLD inhibitors, i.e. 1-propranol or 1-butanol, the compound to be tested comprises at least one primary hydroxyl group attached to a physiologically acceptable carrier moiety through a linear alkyl chain spacer group n carbon atoms in length wherein n is an integer from 3 to 10. Altematively, the spacer group may be a linear group n heteroatoms atoms in length wherein n is an integer from 3 to 20. Alternatively, the primary hydroxyl group may be replaced by a chemically analogous group, such as a primary sulfhydryl group.

The carrier moiety of the compound to be tested may be any physiologically acceptable molecular species, including an sulfhydryl, a hydroxyl, an amino, a cyano, a phosphate, a thiophbsphate, a mercapto, a lower alkyl, a lower alkenyl, an aromatic ring, a heterocyclic ring, a saccharide, a polysaccharide, a glycosaminoglycan, a salicylate, an amino acid, a peptide, a glyceride, a glycol, a lipid, or combinations thereof.

One skilled in the art and acquainted with the invention and examples disclosed herein, may readily apply the above method for the identification of a compound or composition which is an inhibitor or activator of MMP expression.

METHOD FOR DETECTING AN MMP OR PLD ASSOCIATED DISORDER—The association between PLD activity and MMP expression can also be exploited in a detection method designed to identify and quantify the relationship between the activity of PLD and the expression of MMP's in a biological tissue. Such a method, as described below, has multiple utilities in the diagnosis and management of disease wher in dysregulation of MMP expression or of PLD activity is a feature of the pathophysiology. One utility is as a diagnostic procedure for the presence of a cell proliferative disorder, or for quantifying the potential for the development of a cell proliferative disorder, in a selected group of cells or tissues. Measurement of MMP expression in association with the level of PLD activity provides information regarding the physiological state of cells not obtainable by measuring MMP expression alone. In the method, PLD activity is modified through alternately exposing of the biological cell or tissue preparation to inhibitors of the enzyme, thereby allowing for the measurement of MMP expression at varying levels of PLD activity. The extent of MMP suppression thus obtained provides information regarding the nature of the MMP dysregulation and its association with the disease state. Thus the method allows for determining the extent to which the MMP-associated disease state, or tendency to MMP-associated disease, is driven by the activity of PLD. Such information is useful not only in characterization of the MMP-associated disease state in a more definitive way, but facilitates the ability to assess the therapeutic efficacy of a given PLD inhibitor with regard to the specific disease state or patient subjected to the diagnostic test. The test may be applied not only to tissues wherein disease is overt but to biological samples wherein it is desirable to define the relationship between MMP expression and PLD activity, particularly since PLD-driven MMP expression may be detectable before overt signs of disease develop. Thus the diagnostic method may be applied to the cells or tissues of a subject for the purpose of screening for a disease, or for the tendency to develop a disease, such as desirable in the case of a cell proliferative disorder, a cancerous tumor, or an invasive cancer. A particularly useful mode of the invention would be to determine the potential within a tissue preparation excised or biopsies from a patient to develop progressively invasive disease, as well as to assess the potential for response to therapeutic agents. The incorporation of MMP inducing agents within the process provides for application of the detection system to a wider range of cell types and tissues as well as serving to enhance the sensitivity of the assay in a quantitative aspect. Similarly the incorporation of PLD inducing agents within the process provides for application of the detection system to identify and quantify cell types and tissues which express aberrant levels of this enzyme, in one or more pro-enzyme or isozyme forms, such as may be the case in pathological disorders of MMP dysregulation. The system is useful for application to the assessment or diagnosis of cell proliferative disorders, including but not only, epithelial hyperplasia or mucosal hyperplasia particularly in secretory tissues such as in the alimentary tract, respiratory tract or in mammary tissue, in particular adenocarcinomas, non-small cell lung cancer, colorectal carcinoma and breast cancer. In addition CNS proliferative disorders are also objects of the method, wherein cell proliferative disorders known to manifest aberrant MMP or MMP-9 expression, are involved such as but not only malignant gliomas and neuroblastomas. Other nervous system tissue applicable to use as samples in the method are spinal fluid, wherein the object of the method is to assess for MMP-associated disease as in CNS malignancies and in demyelinative disorders such as multiple sclerosis or Guillain Barre Syndrome.

The system for detection of cell proliferative disorders based upon identifying aberrant levels of MMP expression provided by the invention comprises several steps, which may be performed in the following order selecting cells from a cell type which expresses MMP or which may be induced to express MMP; contacting said cells with an inducing agent of MMP expression; determining the level of MMP expression of said cells; contacting said cells with a composition comprising at least one phospholipase D effector; further contacting said cells with an inducing agent of MMP expression; contacting said cells with a composition comprising at least one PLD effector; further determining the level of MMP expression of said cells; diagnosing the presence of a MMP-related disease based upon an at least 1% suppression of MMP expression associated with exposure to the PLD effector.

The system for detection of cell proliferative disorders based upon identifying aberrant levels of PLD expression provided by the invention comprises several steps, which may be performed in the following order: selecting cells from a cell type which expresses PLD or which may be induced to express PLD; contacting said cells with an inducing agent of MMP expression; determining the level of PLD expression of said c lls; contacting said cells with a composition comprising at least one phospholipase D effector; further contacting said cells with an inducing agent of MMP expression; contacting said cells with a composition comprising at least one PLD effector; further determining the level of PLD expression of said cells; diagnosing the presence of a PLD-related disease based upon at least 1% suppression of PLD expression associated with exposure to the PLD effector.

One skilled in the art and acquainted with the invention and examples disclosed herein, may readily apply the above method for identifying an aberrant level of MMP expression or of PLD expression in a cell or tissue and for quantifying the level of aberrant expression for the purpose of assessing the presence of a MMP or PLD related disorder of a cell or tissue.

### EXAMPLES

### Methods and Materials

MMP-9 secretion and activity assay Before assay, the cell medium was replaced with fresh DMEM, 0.1% BSA containing, unless otherwise described, 100 nM PMA. In experiments involving inhibition with BFA, it was added 30 min before induction with PMA. Medium samples were collected after 7.5 h and loaded with non-reducing sample buffer (2% SDS, 10% glycerol in 62.5 mM Tris pH 6.8) on zymogram gels. Before developing, gels were rinsed for 30 min in renaturing buffer and then 30 min in developing buffer at room temperature. Gels were incubated in fresh developing buffer for 18 h at 37° C. MMP-9 activity was indicated by the clear 92 kDa band that appeared after staining with Coomassie brilliant blue and removing excess dye by an 18 h rinse in water. Gels were dried and scanned and then the image was inverted (clear to black and black to clear).

In vivo PLD assay Serum-deprived cells were labeled for 18 h with 1 μCi/ml [³H]myristic acid and were washed with DMEM, 0.1% BSA. Following 20 min preincubation in DMEM, 0.1% BSA, 0.3% 1-butanol, the cells were stimulated with, unless otherwise described, 100 nM PMA for 20 min. Cells were washed with phosphate saline buffer (PBS) (2.68 mM KC1, 1.47 mM KH₂PO₄, 8.05 mM Na₂PO₄, 137 mM NaC1), 0.1% BSA, scraped with 1 ml ice cold CH₃OH and transferred into glass tubes. CHC1₃ and 0.1 N HC1 were added to a final ratio of 1:1:1. The lipid-containing lower phase was collected, dried and dissolved in 30 μl CH₃OH, CHC1₃ (1:1). The samples were loaded on a TLC plate which was developed in the lower phase of H₂O, ethylacetate, acetic acid and iso-octane (100:110:20:50 respectively). Tritiated phosphatidylbutanol (PdtBut) was measured after scraping the band corresponding to the PtdBut standard (Avanti Polar Lipids).

Cell lysis and immunoblotting Overnight serum-deprived cells in 60 mm plates were washed with PBS and then lysed with 750 μl RIPA⁻ buffer (1% NP-40, 0.1% SDS, 100 mM NaF, 10 mM Na pyrophosphate, 2.5 mM Na₃VO₄ in PBS). RIPA⁻ soluble fraction was separated from the non-solube fraction by 15 min centrifugation in a bench top microcentrifuge at 14,000 rpm at 4° C. Protein samples (15 μg) were analyzed after SDS-polyacrylamide gel electrophoresis (SDS-PAGE) by immunoblotting with rabbit anti MMP-9 or anti actin diluted according to manufacturer instructions in PBS, 0.5% Tween 20 and 5% dry milk powder and with horseradish peroxidase-labeled secondary antibody. Immunoreactive bands were visualized by the enhanced chemiluminescence (ECL) reaction.

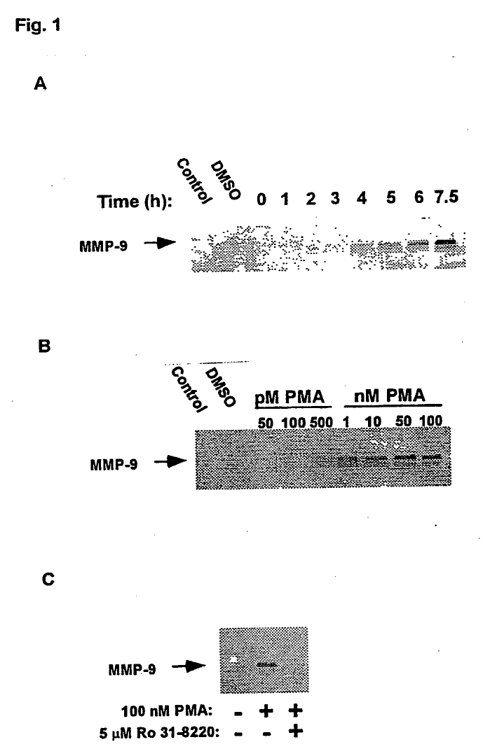

Example 1. MMP-9 Secretion From HT 1080 Cells in Response to PMA Treatment. To establish the role of PLD and PA in the induction of MMP-9 secretion from HT 1080 cells, PMA stimulated MMP-9 secretion from HT 1080 cells is demonstrated. The cells were treated with PMA for various times, and then medium samples were assayed for collagenolytic activity. FIG. 1A illustrates that MMP-9 activity accumulates in the medium of cells treated with PMA in a time-dependent manner, whereas no activity is observed in untreated cells or cells treated with dimethyl sulfoxide, the solvent for PMA The effect of PMA is dose-dependent, with secretion being detected with concentrations as low as 0.5-1 nM and reaching a plateau at 50-100 nM PMA (FIG. 1B). Finally, the expected involvement of PKC in the effect is confirmed by the exhibited inhibition of secretion in cells treated with the PKC blocker Ro 31-8220 (FIG. 1C).

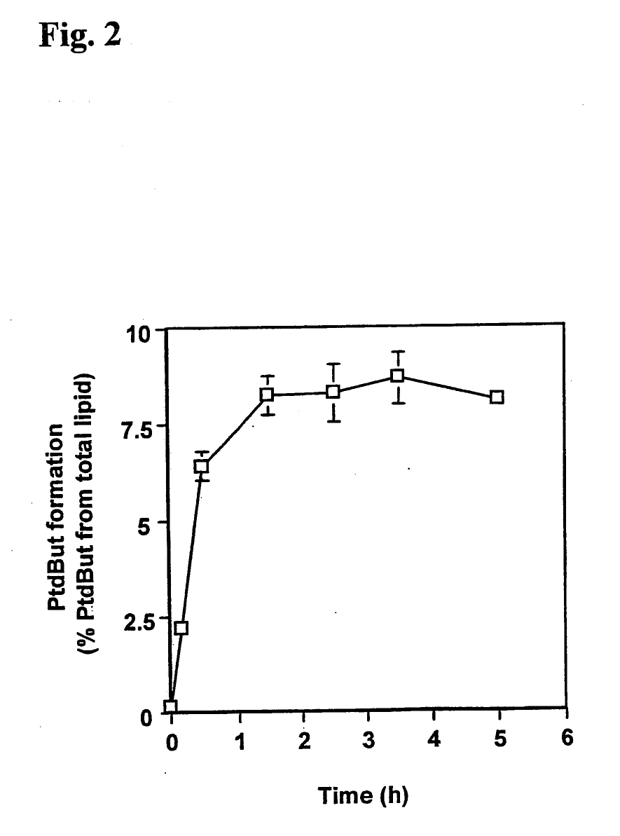

Example 2. PLD Activation is Involved in the Induction of MMP-9 Secretion by PMA. PKC isozymes have a broad spectrum of effects and among these is the activation of PLD (12). Therefore, the involvement of PLD in the stimulation of MMP-9 secretion by PMA is demonstrated through assessing PMA induced PLD activation in HT 1080 cells. HT 1080 cells prelabeled with [3H]myristate were stimulated with PMA for various times, and PLD activity was measured by the formation of [3H]phosphatidylbutanol (PtdBut) from 1-butanol. As shown in FIG. 2, PtdBut formation is rapidly induced and reaches a maximum after 90 min. The response is detectable with 1 nM PMA and maximal at 100 nM PMA (data not shown).

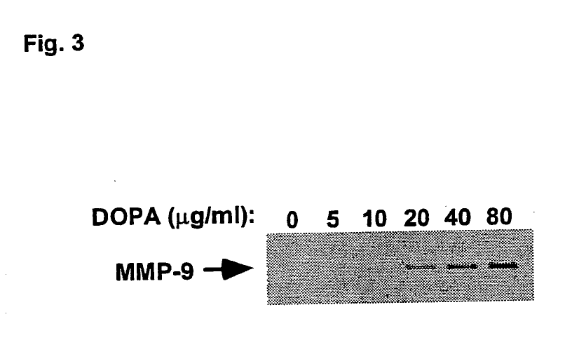

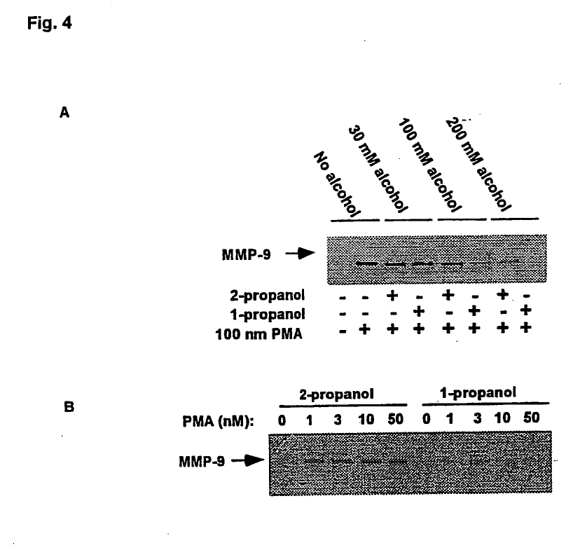

Although the preceding experiments show that PLD is activated in PMA-treated HT 1080 cells, they provide only circumstantial evidence that PLD is involved in the secretory pathway of matrix metalloprotein. To confirm the direct role of PLD, the cells were treated with various concentrations of a short-chain (dioctanoyl) PA (DDPA or DOPA). FIG. 3 shows that DDPA induces MMP-9 secretion in a dose-dependent manner with secretion reaching a plateau at 80 μg/ml. A role for PLD in the pathway leading to MMP-9 secretion is further supported upon incorporating the PLD inhibitor 1-propanol to reduce PA production by directing the formation of phosphatidylpropanol, rather than of PA, through the transphosphatidylation reaction. Cells were first treated with various concentrations of 1-propanol or, 2-propanol as a control, and then stimulated with PMA. Medium samples were collected after 7.5 h and analyzed for MMP-9 secretion. Secretion is inhibited by 100 mM 1-propanol but not by the same concentration of 2-propanol (FIG. 4A). At 200 mM, both alcohols are inhibitory, but the effect of 1-propanol is an almost absolute suppression of MMP-9 expression. In further experiments, MMP-9 secretion was assessed from cells treated with 133 mM of either 1-propanol or 2-propanol prior to stimulation with various PMA concentrations. Although in the presence of 2-propanol, MMP-9 secretion in response to PMA continues to be dose-dependent, in the presence of 1-propanol secretion is almost totally suppressed at all doses of the tumor promotor (FIG. 4B). These results demonstrate that PLD activity and the intracellular accumulation of PA are critical stages in the pathway of PKC-dependent MMP-9 secretion.

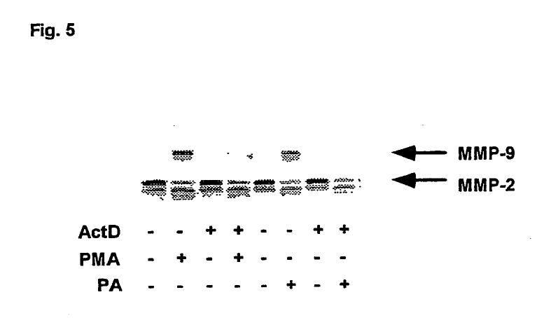

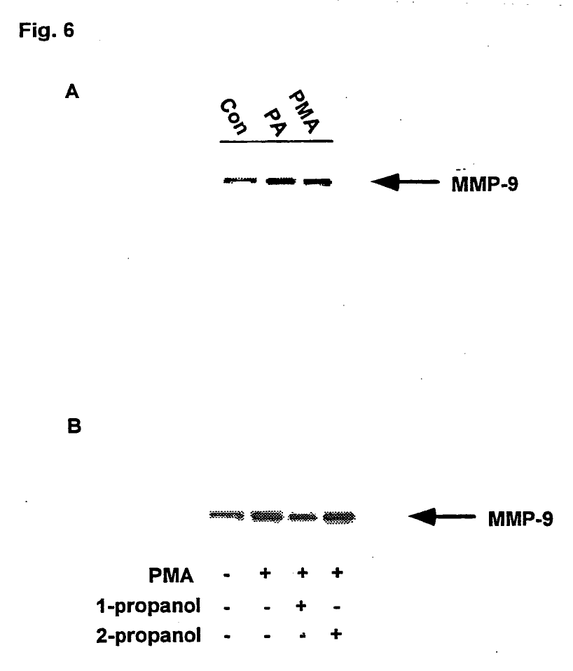

Example 3. PKC Induces MMP-9 expression Via PLA Activation. To demonstrate the mechanism of the effect of PA on MMP-9 secretion, and knowing that PKC upregulates gene expression, we tested the possibility that PA has a similar role. The first hint that PA is involved in MMP-9expression came from experiments employing ActD as a specific inhibitor of protein synthesis. HT 1080 cells were subjected to ActD prior to the induction with PA ActD fully decreased MMP-9 secretion suggesting (FIG. 5) that secretion of MMP-9 upon PA stimulation is dependent upon de novo protein synthesis. In cells exposed to primary alcohol and then treated with PMA, MMP-9 expression is suppressed in cells pretreated with the primary alcohol 1-propanol (FIG. 6), demonstrating that PKC-dependent MMP-9 expression is mediated by PLD activation mediated through the intracellular level of PA.

### References

1. Kanfer, J. N. (1980). The base exchange enzymes and phospholipase D of mammalian tissue. Can. J. Biochem. 58, 1370-1380.

2. Ktistakis, N. T., H. A. Brown, M. G. Waters, P. C. Stemweis, and M. G. Roth. (1996). Evidence that phospholipase D mediates ADP ribosylation factor-dependent formation of Golgi coated vesicles. J. Cell Biol. 134, 295-306.

3. Bi, K., M. G. Roth, and N. T. Ktistakis. (1997). Phosphatidic acid formation is required for transport from the endoplasmic reticulum to the Golgi complex. Curr. Biol. 7, 301-307.

4. Chen, Y. G., A Siddhanta, C. D. Austin, S. M. Hammond, T. C. Sung, M. A. Frohman, A. J. Morris, and D. Shields. (1997). Phospholipase D stimulates release of nascent secretory vesicles from the trans-Golgi network. J. Cell Biol. 138, 495-504.

5. Siddhanta, A. and D. Shields. (1998). Secretory vesicle budding from the trans-golgi network is mediated by phosphatidic acid levels. J. Biol. Chem. 273, 17995-17998.

6. Liotta, L. A., K Tryggvason, S. Garbisa, P. Gehron-Robey, and Abe, S. (1981). Partial purification and characterization of a neutral protease which cleaves type IV collagen. Biochem. 20, 100-104.

7. Liotta, L. A., Abe, S., Gehron-Robey, P., and Martin, G. R. (1979). Preferential digestion of basement membrane collagen by an enzyme derived from a metastatic murine tumor. Proc. Natl. Acad. Sci U.S.A. 76, 2268-2272.

8. Ballin, M., Gomez, D. E., Sinha, C. C., and Thorgeirsson, U. P. (1988). Ras oncogene mediated induction of a 92 kDa metalloproteinase; strong correlation with the malignant phenotype. Biochem. Biophys. Res. Commun. 154, 832-838.

9. Mackay A. R., Ballin, M., Pelina, M. D., Farina, A. R., Nason, A. M., Hartzler, J. L., and Thorgeirsson, U. P. (1992). Effect of phorbol ester and cytokines on matrix metalloproteinase and tissue inhibitor of metalloproteinase expression in tumor and normal cell lines. Invasion Metastasis 12, 168-84.

10. Okada, Y., Gonoji, Y., Naka, K., Tomita, K., Nakanishi, I., Iwata, K., Yamashita, K., and Hayakawa, T. (1992). Matrix metalloproteinase 9 (92-kDa gelatinase/type IV collagenase) from HT 1080 human fibrosarcoma cells. Purification and activation of the precursor and enzymic properties. J. Biol. Chem. 267, 21712-21719.

11. Goldberg, G. I., Collier, I. E., Eisen, A. Z., Grant, G. A, Marmer, B. L., and Wilhelm, S. M. (1992). Mosaic structure of the secreted ECM metalloproteases and interaction of the type IV collagenases with inhibitors. Matrix Suppl. 1, 25-30.

12. Exton, J. H. (1997). New developments in phospholipase D. J Biol Chem. 272, 15579-155782.

13. Kaur, M., Kaul, D., and Sobti RC (1998).

Receptor-Ck-dependent regulation of genes involved in the cell cycle. Mol Cell. Biochem. 181, 137-142.

14. Cano, E., Munoz-Femandez, M. A., Fresno, M. (1992). Regulation of interleukin-2 responses by phosphatidic acid. Eur. J. Immunol. 22, 1883-1889.

15. Kim, T. S., and Kim, Y. B. (1999). Correlation between expression of matrix metalloproteinase-2 (MMP-2), and matrix metalloproteinase-9 (MMP-9) and angiogenesis in colorectal adenocarcinoma. J. Korean Med. Sci. 14, 263-270.

16. Forsyth, P. A., Wong, H., Laing, T. D., Rewcastle, N. B., Morris, D. G., Muzik, H., Leco, K J., Johnson, R. N., Brasher, P. M., Sutherland, G., and Edwards, D. E. (1999). Gelatinase-A (MMP-2), gelatinase-B (MMP-9) and membrane type matrix metalloproteinase-1 (MT1-MMP) are involved in different aspects of the pathophysiology of malignant gliomas. Br. J. Cancer. 79, 1828-1835.

17. Ribatti, D., Alessandri, G., Vacca, A., lurlaro, M., and Ponzoni, M. (1998). Human neuroblastoma cells produce extracellular matrix degrading enzymes, induce endothelial cell proliferation and angiogenesis in vivo. Int. J. Cancer 77, 449-454.

18. Tolnay, E., Wiethege, T., Kuhnen, C., Wulf, M., Voss, B., and Muller, K. M. (1997). Expression of type IV collagenase correlates with the expression of vascular endothelial growth factor in primary non-small cell lung cancer. J. Cancer Res. 123, 652-658.

19. Nielsen, B. S., Sehested, M., Kjeldsen,. L., Borregaard, N., Rygaard, J., and Dano, K (1997). Expression of matrix metalloproteinase9 in vascular pericytes in human breast cancer. Lab. Invest. 77, 345-355.

20. Andrews, B, et al. (2000). Direct Inhibition of In Vitro PLD Activity by 4-(2-)Aminoethyl)-Benzenesulfonyl Fluoride. BBRC. 273, 302-311.

What we claim is:

1. A method of suppressing the level of MMP expression of a cell comprising the steps of contacting a cell with an effective amount of a phospholipase D inhibitor, thereby reducing the level of MMP expression of said cell.

2. A method of claim 1, wherein the phospholipase D inhibitor is a compound comprising at least one primary hydroxyl group conjugated to a physiologically acceptable moiety through a linear spacer group n carbon atoms or n heteroatoms atoms in length wherein n is an integer from 3 to 20.

3. A method of claim 1, wherein the phospholipase D inhibitor is a compound selected from the group consisting of 1-propanol or 1-butanol.

4. A method of claim 1, wherein the phospholipase D inhibitor is a compound comprising at least one primary sulfhydryl group conjugated to a physiologically acceptable moiety through a linear spacer group n carbon atoms or n heteroatoms atoms in length wherein n is an integer from 3 to 20.

5. A method of claim 1, wherein the phospholipase D inhibitor is a compound selected from the group consisting of 1-propanthiol or 1-butanthiol.

6. A method of claim 1, wherein the phospholipase D inhibitor is a serine protease inhibitor.

7. A method of claim 1, wherein the phospholipase D inhibitor is 4-(2-aminoethyl)-benzenesulfonyl fluoride.

8. A method of claim 1, wherein the phospholipase D inhibitor is a serine protease inhibitor, wherein said inhibitor is conjugated to a physiologically acceptable moiety through a linear spacer group n carbon atoms or n heteroatoms atoms in length, wherein n is an integer from 0 to 20, wherein said spacer group is covalently attached to a nitrogen atom of said inhibitor.

9. The method of claim 8, wherein said phospholipase D inhibitor is 4-(2-aminoethyl)-benzenesulfonyl fluoride.

10. The method of claims 8 or 9, wherein said physiologically acceptable acceptable moiety is a fatty acid, lipid, phospholipid, or thiophospholipid moiety through a linear spacer group n carbon atoms or n heteroatoms atoms in length, wherein n is an integer from 0 to 20, wherein said spacer group is covalently attached to a nitrogen atom of said inhibitor and to an atom said fatty acid, lipid, phospholipid or thiophospholipid moiety.

11. A method of claims 2, 4, 8, or 9, wherein the physiologically acceptable conjugated moiety is an atom or chemical group selected from the list consisting of hydrogen, halogens, hydroxyl, sulfhydryl, amino, cyano, nitro, phosphate, thiophosphate, mercapto, lower alkyl, lower alkenyl, aromatic rings, heterocyclic rings, heterocyclic aromatic rings, carboxyl, cycloalkyl, cycloalkylalkyl, alkyloxycarbonylalkanoyl, alkyloxycarbonyl, alkanoyl, cycloalkylcarbonyl, heterocycloalkylcarbonyl arylalkyloxycarbonyl, carbamoyl, alkylcarbamoyl, dialkylcarbamoyl, arylcarbamoyl, arylalkylcarbamoyl, arylalkanoyl, aroyl, alkylsulfonyl, dialkylaminosulfonyl, arylsulfonyl, heteroarylsulfonyl, alkyl cycloalkyl, cycloalkylalkyl, heteroaryl, heteroarylalkyl, aryl, arylalkyl, alkoxy, alkylsulfonyl, an arylsulfonyl, saccharides, polysaccharides, glycosaminoglycans, salicylates, steroids, hydroxysteroids, purines, pyrimidines, nucleosides, amino acids, peptides, glycerides, poly-glycerides, glycols, polyglycols, lipids, phospholipids, thiophospholipids, individual isomers, and combinations thereof.

12. A method of claims 2, 4, 8, 9, 10, or 11, wherein the physiologically acceptable conjugated moiety is an inhibitor, an activator, or a substrate of phospholipase D.

13. A method of claims 2, 4, 8, 9, 10, 11, or 12, wherein the physiologically acceptable conjugated moiety enhances the permeability of said inhibitor through biological membranes.

14. A method of claims 1 to 13, wherein said cell is characterized by a cell tissue type selected from the group consisting of epithelium, colon epithelium, glial cells, astrocytes, endotracheal epithelium, and breast epithelium.

15. A method of claims 1 to 13, wherein said cell is characterized by a cancer cell tissue type selected from the group consisting of colorectal adenocarcinoma, malignant gliomas, neuroblastoma, non-small cell lung cancer, and breast cancer.

16. A method for identifying effector compounds of PLD activity, comprising the steps of:
(i) selecting cells from a cell type which expresses MMP or which may be induced to express MMP;
(ii) contacting said cells with an inducing agent of MMP expression;
(iii) determining the level of MMP expression of said cells;
(iv) contacting said cells with a compound initially unknown to affect PLD activity;
(v) determining the level of MMP expression of said cells;
(vi) comparing the determination made in step (iii) with the determination in step (v);
(vii) identifying a compound as an effector compound of PLD activity based on at least a 1% difference as determined in step (vi).

17. A method for identifying effector compounds of MMP expression of cells, comprising the steps of:
(i) selecting cells from a cell type which expresses MMP or which may be induced to express MMP;
(ii) contacting said cells with an inducing agent of MMP expression;
(iii) determining the level of MMP expression of said cells;
(iv) contacting said cells with a phospholipase D effector compound;
(v) contacting said cells with a compound unknown to affect MMP expression;
(vi) determining the level of MMP expression of said cells;
(vii) comparing the determination made in step (iii) with the determination in step (vi);
(viii) identifying a compound as an effector compound of MMP expression based on at least a 1% differ nce as determined in step (vii).

18. A method of claims 16 or 17, wherein the compound of step (iv) comprises at least one primary hydroxyl group conjugated to a physiologically acceptable moiety through a linear spacer group n carbon atoms or n heteroatoms atoms in length wherein n is an integer from 3 to 20.

19. A method of claims 16 or 17, wherein the compound of step (iv) is selected from the group consisting of 1-propanol or 1-butanol.

20. A method of claims 16 or 17, wherein the compound of step (iv) comprises at least one primary sulfhydryl group attached to a physiologically acceptable carrier moiety through a linear spacer group n carbon atoms or n heteroatoms atoms in length wherein n is an integer from 3 to 20.

21. A method of claims 16 or 17, wherein the compound of step (iv) is selected from the group consisting of 1-propanthiol or 1-butanthiol.

22. A method of claims 16 or 17, wherein the compound of step (iv) comprises a PLD inhibitor conjugated to a physiologically acceptable moiety, wherein said moiety is an atom or chemical group selected from the list consisting of hydrogen, halogens, hydroxyl, sulfhydryl, amino, cyano, nitro, phosphate, thiophosphate, mercapto, lower alkyl, lower alkenyl, aromatic rings, heterocyclic rings, heterocyclic aromatic rings, carboxyl, cycloalkyl, cycloalkylalkyl, alkyloxycarbonylalkanoyl, alkyloxycarbonyl, alkanoyl, cycloalkylcarbonyl, heterocycloalkylcarbonyl arylalkyloxycarbonyl, carbamoyl, alkylcarbamoyl, dialkylcarbamoyl, arylcarbamoyl, arylalkylcarbamoyl, arylalkanoyl, aroyl, alkylsulfonyl, dialkylaminosulfonyl, arylsulfonyl, heteroarylsulfonyl, alkyl cycloalkyl, cycloalkylalkyl, heteroaryl, heteroarylalkyl, aryl, arylalkyl, alkoxy, alkylsulfonyl, an arylsulfonyl, saccharides, polysaccharides, glycosaminoglycans, salicylates, steroids, hydroxysteroids, purines, pyrimidines, nucleosides, amino acids, peptides, glycerides, poly-glycerides, glycols, polyglycols, lipids, individual isomers and combinations thereof.

23. The method of claims 16 or 17, wherein the compound of step (iv) is a serine protease inhibitor.

24. The method of claims 16 or 17, wherein the compound of step (iv) is 4-(2-aminoethyl)-benzenesulfonyl fluoride.

25. The method of claims 23 or 24, wherein the compound of step (iv) is a serine protease inhibitor conjugated to a physiologically acceptable moiety through a linear spacer group n carbon atoms or n heteroatoms atoms in length, wherein n is an integer from 0 to 20, wherein said spacer group is covalently attached to a nitrogen atom of said inhibitor.

26. The method of claim 25, wherein said serine protease inhibitor is 4-(2aminoethyl)-benzenesulfonyl fluoride.

27. The method of claims 25 or 26, wherein said physiologically acceptable acceptable moiety is a fatty acid, lipid, phospholipid, or thiophospholipid moiety through a linear spacer group n carbon atoms or n heteroatoms atoms in length, wherein n is an integer from 0 to 20, wherein said spacer group is covalently attached to a nitrogen atom of said inhibitor and to an atom said fatty acid, lipid, phospholipid or thiophospholipid moiety.

28. A method of claims 18, 20, 22, 25, or 27, wherein the conjugated moiety is an inhibitor, an activator, or a substrate of phospholipase D.

29. A method of claims 16 to 28, wherein said MMP inducing agent is an activator of phosphokinase C activity.

30. A method of claims 16 to 29, wherein said MMP inducing agent is selected from the group consisting of phorbol esters, diacylglycerols, or thapsigarin.

31. A method of claims 16 to 30, wherein the said cells are selected from the group consisting of epithelium, colon epithelium, glial cells, astrocytes, endotracheal epithelium, and breast epithelium.

32. A method of claims 16 to 30, wherein said cells selected from the group consisting of colorectal adenocarcinoma, malignant gliomas, neuroblastoma, non-small cell lung cancer, and breast cancer.

33. A compound or composition identified by the method of claims 16 to 32.

34. A method of identifying a cell or biological tissue exhibiting aberrant levels of MMP expression comprising the steps of:
(i)selecting cells from a cell or tissue type which expresses MMP or which may be induced to express MMP;
(ii) contacting said cells with an inducing agent of MMP expression;
(iii) determining the level of MMP expression of said cells;
(iv) contacting said cells with a phospholipase D effector compound;
(v) determining the level of MMP expression of said cells;
(vi) determining the level of MMP expression of said cells or said biological tissue preparation based on comparing the determination made in step (iii) with the determination in step (v). (vii) identifying a cell or tissue as exhibiting aberrant levels of MMP expression based on at least a 1% difference as determined in step (vi).

35. A method of identifying a cell or biological tissue exhibiting aberrant levels of PLD expression comprising the steps of:
(i) selecting cells from a cell or tissue type which expresses PLD or which may be induced to express PLD;
(ii) contacting said cells with an inducing agent of PLD expression;
(iii) determining the level of PLD expression of said cells;
(iv) contacting said cells with a phospholipase D effector compound;
(v) determining the level of PLD expression of said cells;
(vi) determining the level of PLD expression of said cells or said biological tissue preparation based on comparing the determination made in step (iii) with the determination in step (v). (vii) identifying a cell or tissue as exhibiting aberrant levels of PLD expression based on at least a 1% difference as determined in step (vi).

36. A method of claims 34 or 35, wherein the compound of step (iv) comprising at least one primary hydroxyl group conjugated to a physiologically acceptable moiety through a linear spacer group n carbon atoms or n heteroatoms atoms in length wherein n is an integer from 3 to 20.

37. A method of claims 34 or 35, wherein the compound of step (iv) is selected from the group consisting of 1-propanol or 1-butanol.

38. A method of claims 34 or 35, wherein the compound of step (iv) comprising at least one primary sulfhydryl group attached to a physiologically acceptable carrier moiety through a linear spacer group n carbon atoms or n heteroatoms atoms in length wherein n is an integer from 3 to 20.

39. A method of claims 34 or 35, wherein the compound of step (iv) is selected from the group consisting of 1-propanthiol or 1butanthiol.

40. A method of claims 34 or 35, wherein the physiologically acceptable conjugated moiety is an atom or chemical group selected from the list consisting of hydrogen, halogens, hydroxyl, sulfhydryl, amino, cyano, nitro, phosphate, thiophosphate, mercapto, lower alkyl, lower alkenyl, aromatic rings, heterocyclic rings, heterocyclic aromatic rings, carboxyl, cycloalkyl, cycloalkylalkyl, alkyloxycarbonylalkanoyl, alkyloxycarbonyl, alkanoyl, cycloalkylcarbonyl, heterocycloalkylcarbonyl arylalkyloxycarbonyl, carbamoyl, alkylcarbamoyl, dialkylcarbamoyl, arylcarbamoyl, arylalkylcarbamoyl, arylalkanoyl, aroyl, alkylsulfonyl, dialkylaminosulfonyl, arylsulfonyl, heteroarylsulfonyl, alkyl cycloalkyl, cycloalkylalkyl, heteroaryl, heteroarylalkyl, aryl, arylalkyl, alkoxy, alkylsulfonyl, an arylsulfonyl, saccharides, polysaccharides, glycosaminoglycans, salicylates, steroids, hydroxysteroids, purines, pyrimidines, nucleosides, amino acids, peptides, glycerides, poly-glycerides, glycols, polyglycols, lipids, individual isomers and combinations thereof.

41. The method of claims 34 or 35, wherein the compound of step (iv) is a serine protease inhibitor.

42. The method of claim 41, wherein the compound of step (iv) is 4-(2-aminoethyl)-benzenesulfonyl fluoride.

43. The method of claim 41, wherein the compound of step (iv) is a serine protease inhibitor conjugated to a physiologically acceptable moiety through a linear spacer group n carbon atoms or n heteroatoms atoms in length, wherein n is an integer from 0 to 20, wherein said spacer group is covalently attached to a nitrogen atom of said inhibitor.

44. The method of claim 43, wherein said serine protease inhibitor is 4-(2-aminoethyl)-benzenesulfonyl fluoride.

45. The method of claims 43 or 44, wherein said physiologically acceptable acceptable moiety is a fatty acid, lipid, phospholipid, or thiophospholipid moiety through a linear spacer group n carbon atoms or n heteroatoms atoms in length, wherein n is an integer from 0 to 20, wherein said spacer group is covalently attached to a nitrogen atom of said inhibitor and to an atom said fatty acid, lipid, phospholipid or thiophospholipid moiety.

46. The method of claims 34 to 40, wherein the conjugated moiety is an inhibitor, an activator, or a substrate of phospholipase D.

47. The method of claims 34 to 40, wherein said MMP inducing agent is an activator of phosphokinase C activity.

48. The method of claim 34 to 40, wherein said PLD inducing agent is selected from the group consisting of phorbol esters, diacylglycerols, or thapsigarin.

49. The method of claims 34 to 40, wherein said cells are selected from the group consisting of epithelium, colon epithelium, glial cells, astrocytes, endotracheal epithelium, and breast epithelium.

50. The method of claims 34 to 40, wherein said cells selected from the group consisting of colorectal adenocarcinoma, malignant gliomas, neuroblastoma, non-small cell lung cancer, and breast cancer.

51. A method for treating a subject afflicted with a cell proliferative disorder, comprising the steps of administering to a subject an effective amount of a phospholipase D inhibitor, thereby treating the subject afflicted with a cell proliferative disorder.

52. A method for treating a subject afflicted with a demyelinative disorder, comprising the steps of administering to a subject an effective amount of a phospholipase D inhibitor, thereby treating the subject afflicted with a demyelinative disorder.

53. A method of claims 51 or 52, wherein the phospholipase D inhibitor is a compound comprising at least one primary hydroxyl group conjugated to a physiologically acceptable moiety through a linear spacer group n carbon atoms or n heteroatoms atoms in length wherein n is an integer from 3 to 20.

54. A method of claim 51 or 52, wherein the phospholipase D inhibitor is a compound selected from the group consisting of 1-propanol or 1-butanol.

55. A method of claims 51 or 52, wherein the phospholipase D inhibitor is a compound comprising at least one primary sulfhydryl group conjugated to a physiologically acceptable moiety through a linear spacer group n carbon atoms or n heteroatoms atoms in length wherein n is an integer from 3 to 20.

56. A method of claims 51 or 52, wherein the phospholipase D inhibitor is a compound selected from the group consisting of 1-propanthiol or 1-butanthiol.

57. A method of claims 51 or 52, wherein the physiologically acceptable conjugated moiety is an atom or chemical group selected from the list consisting of hydrogen, halogens, hydroxyl, sulfhydryl, amino, cyano, nitro, phosphate, thiophosphate, mercapto, lower alkyl, lower alkenyl; aromatic rings, heterocyclic rings, heterocyclic aromatic rings, carboxyl, cycloalkyl, cycloalkylalkyl, alkyloxycarbonylalkanoyl, alkyloxycarbonyl, alkanoyl, cycloalkylcarbonyl, heterocycloalkylcarbonyl arylalkyloxycarbonyl, carbamoyl, alkylcarbamoyl, dialkylcarbamoyl, arylcarbamoyl, arylalkylcarbamoyl, arylalkanoyl, aroyl, alkylsulfonyl, dialkylaminosulfonyl, arylsulfonyl, heteroarylsulfonyl, alkyl cycloalkyl, cycloalkylalkyl, heteroaryl, heteroarylalkyl, aryl, arylalkyl, alkoxy, alkylsulfonyl, an arylsulfonyl, saccharides, polysaccharides, glycosaminoglycans, salicylates, steroids, hydroxysteroids, purines, pyrimidines, nucleosides, amino acids, peptides, glycerides, poly-glycerides, glycols, polyglycols, lipids, individual isomers and combinations thereof.

58. The method of claims 51 or 52, wherein the phospholipase D inhibitor is a serine protease inhibitor.

59. The method of claims 51 or 52, wherein the phospholipase D inhibitor is 4-(2-aminoethyl)-benzenesulfonyl fluoride.

60. The method of claims 51 or 52, wherein the c phospholipase D inhibitor is a serine protease inhibitor conjugated to a physiologically acceptable moiety through a linear spacer group n carbon atoms or n heteroatoms atoms in length, wherein n is an integer from 0 to 20, wherein said spacer group is covalently attached to a nitrogen atom of said inhibitor.

61. The method of claims 51 or 52, wherein said phospholiapse D inhibitor is 4-(2-aminoethyl)-benzenesulfonyl fluoride.

62. The method of claim 60 or 61, wherein said physiologically acceptable acceptable moiety is a fatty acid, lipid, phospholipid, or thiophospholipid moiety through a linear spacer group n carbon atoms or n heteroatoms atoms in length, wherein n is an integer from 0 to 20, wherein said spacer group is covalently attached to a nitrogen atom of said inhibitor and to an atom said fatty acid, lipid, phospholipid or thiophospholipid moiety.

63. A method of claims 51 to 62, wherein the conjugated moiety is an inhibitor, an activator, or a substrate of phospholipase D.

64. A method of claims 51 to 62, wherein the phospholipase D inhibitor is a compound which suppresses the level of MMP expression of a cell.

65. A method of claims 41 to 62, wherein the cell proliferative disorder is characterized by a cell tissue type selected from the group consisting of epithelium, colon epithelium, glial cells, astrocytes, endotracheal epithelium, and breast epithelium.

66. A method of claims 51 to 62, wherein the cell proliferative disorder is characterized by a cancer cell tissue type selected from the group consisting of colorectal adenocarcinoma, malignant gliomas, neuroblastoma, non-small cell lung cancer, and breast cancer.

67. A method of claims 51 to 62, wherein said demyelinative disorder is selected from the group consisting of multiple sclerosis or Guillian Barre Syndrome.
